(19) United States
(12) Patent Application Publication
(10) Pub. No.: US 2005/0251117 A1
(43) Pub. Date: Nov. 10, 2005

(54) Apparatus and method for treating biological external tissue

(76) Inventors: Robert S. Anderson, Livermore, CA (US); Alon Maor, Los Altos, CA (US); Steve Young, Discovery Bay, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/841,273

(22) Filed: May 7, 2004

(51) Int. Cl. A61B 18/20; A61B 18/18

(52) U.S. Cl. 606/9; 607/89; 606/41; 607/101

(57) ABSTRACT

A method and device for treating biological external tissue using at least one energy source. The energy source can be incoherent light, coherent light, a radio frequency, ultrasound, a laser, or any other type of energy that can be applied through the device. The features of various embodiments of the device include the generation of positive pressure and/or negative pressure through one or more pressure conduits, the application of an object within a recess of the device, and measurements through various sensors on the device. These sensors can be monitored and/or controlled through a display element having rows and columns of pixels on the device. The device can be a handheld device or an add-on to existing devices in some embodiments, and can include skin color sensors, temperature sensors, and capacitance sensors.

### FIELD OF THE INVENTION

The present invention relates to methods and devices useful in modification, treatment, destruction, and/or removal of tissue.

### BACKGROUND OF THE INVENTION

Devices utilized in dermatological treatments often incorporate light based energy sources or high frequency rf electrical energy sources. Examples of such devices are described in U.S. Pat. No. 6,511,475. Some devices include both technologies.

A. Lasers and Light-Based Technologies

Lasers and light-based devices have been used for many years in the treatment of dermatological conditions. Soon after the laser was invented in 1957, medical researchers started to explore its use for a wide range of dermatological procedures. In recent years, especially since the mid-90's, the technology has been commercialized into numerous different devices that remove unwanted hair, wrinkles, fine lines and various facial blemishes (“skin rejuvenation”), tattoos, and vascular and pigmented lesions. Because of the short treatment time, virtually no patient “down-time” and fewer side effects, several of these laser- or light-based treatments have become more widely used than the conventional alternatives.

Light energy, when applied directly to the human body, is absorbed by the target chromophore; by the hemoglobin in the blood; the water in the skin; the melanin in the skin; and/or by the melanin in the hair follicles, depending on the wavelength(s) of the light used. Lasers generating different wavelengths of light were found early on to have different properties, each being preferable for specific procedures. In addition to lasers that emit a coherent, monochromatic light, several manufacturers have also introduced devices that emit light of a wide range of wavelengths that practitioners then filter to select the appropriate wavelength for a specific treatment. These “multi-wavelength” or “multi-application” light-based devices have the advantage of performing several different aesthetic treatments, and thus costing the practitioner less than purchasing several lasers individually.

FIG. 1a is a diagram showing the various layers of the skin and potential targets for photo therapy and/or electrical therapy. When light energy first impacts the skin, it encounters the epidermis, the outer most layer of skin. One of the substances that comprise the epidermis is melanin, the brown pigmentation that most of us have in our skin. Darker individuals have more melanin than lighter ones. For very dark individuals, melanin may comprise more than 20% of the epidermis. For light skin individuals, melanin may comprise only 1 to 2% of the epidermis.

Melanocytes in the upper epidermis generate this melanin in response to sunlight. The melanin migrates from the cell and forms a protective umbrella over the fibroblasts and other cells in the skin. The melanin absorbs harmful UVA and UVB radiation that can cause cell damage. It also absorbs visible light, absorbing blue light more than red light.

The epidermis is very thin as it is only 50 to 100 microns in thickness. Consequently, despite the strong absorption by melanin, a reasonable percentage of the light passes through the epidermis into the upper layer of the dermis. For a fair skin person, as little as 15% of the light in the visible portion of the spectrum is absorbed in the epidermis. For a darker person, the percentage absorbed can be more than 50%.

After passing through the epidermis, the light impacts a region called the dermal plexus. This is a thin region at the outer most region of the dermis. It contains a high concentration of small capillary vessels that provide nourishment to the overlying epidermis. The blood in these vessels absorbs between 35% and 40% of the visible portion of the light that impacted the skin.

Clearly for a moderate to dark skin individual, the majority of the visible portion of the spectrum is absorbed in the epidermis and the dermal plexus. Very little energy remains to treat a target located deeper than the dermal plexus.

FIG. 1b shows the percentage of incident energy transmitted, as a function of wavelength, through the epidermis for three different skin types. The figure shows a low percentage of the incident energy in the visible portion of the spectrum is transmitted through the epidermis. The energy not transmitted is absorbed, resulting in a rise in temperature of the epidermis and possibly resulting in the burning of the tissue.

FIG. 1c shows the percentage of incident energy transmitted through the dermal plexus for two different levels of blood concentration (shown as ratios of blood to the rest of the tissue in a given volume). As in the epidermis, the energy not transmitted is absorbed and can produce burning. More importantly, the energy absorbed in the dermal plexus is not available to heat a target such as collagen or tattoo ink that is located beneath the dermal plexus. By reducing the concentration in half, the energy transmitted is doubled.

B. High Frequency rf Electrical Devices

In addition to light based therapies, high frequency rf electrical energy is also becoming common in devices used to treat wrinkles, unwanted hair and unwanted vascular lesions. One of the basic principles of electricity is an electric current passing through a resistive element generates heat in that element. The power dissipated in the element is proportional to the square of the electrical current and also proportional to the resistance of the element. The heat generated is the product of the power times the length of time the power is being dissipated.

A second basic principle of electricity is the electric current seeks the path of least resistance. If two or more such paths exist, the current divides itself proportionally to the resistance of each path. For example, if two such paths exist and one path is twice the resistance of the other, twice the current will pass through the path with the lesser resistance than passes through the path with more resistance. The distribution of power and energy is also in the ratio of the resistances. In the current example, two times the power is dissipated in the lower resistance path than in the higher path. The path with the lesser resistance will heat at twice the rate as the higher resistance path.

High frequency rf energy in dermatology works on the principles described above. In this case, the various tissues and components of the body are the electrical resistors. As the rf current passes through these tissues, energy is dissipated and the temperature of the tissue rises. If the tissue is a blood vessel, it may reach a temperature at which the blood denatures and coagulates. If the tissue is collagen, it may reach a temperature at which the collagen denatures and is destroyed. The body natural immune system removes the destroyed tissue, starting a process to regenerate new tissue.

The electrical resistance of various tissues varies widely. Tissues in the body with relatively high resistance are bone, fat and the outer layer of the epidermis. Tissues with moderate resistance are connective tissue and the dermis. The tissue with the lowest resistance is the blood. When high frequency electricity is used in dermatological applications, it tends to follow the pathways of the blood vessels, avoiding the fatty tissues and connective tissues.

### SUMMARY OF THE DESCRIPTION

There are numerous different embodiments of apparatuses and methods which are described below. The apparatuses are typically (but not necessarily) handheld devices which apply energy (e.g., coherent or incoherent light) from one or more sources in the handheld device. The device may include a negative pressure conduit (e.g., a tube which couples the skin to a vacuum source/pump) which can be used to draw the skin into a region of the device. This will tend to stretch the skin and bring one or more targets (below the surface of the skin) closer to the surface so that these targets receive more incident energy as a result of being closer to the surface.

The device may also include a pixilated display for displaying information (e.g., skin temperature, elapsed treatment time, etc.). The device may also include sensors (e.g., skin temperature sensor and/or skin color sensor) and may also include an object which is used to mechanically push the skin (thereby providing a positive pressure to a portion of the skin). A device may have multiple, different sources of energy. The sources of energy may, for example, be different laser diodes which emit light of different wavelengths. A device may include a pressure conduit which creates a positive pressure (e.g., a pressure above ambient atmospheric pressure). This pressure conduit may, in certain embodiments, be the same conduit which provides a vacuum or it may be a different, separate conduit. It will be appreciated that there are various alternative apparatuses which can have various combinations of the different features. For example, a handheld device may include the following features or a subset of these features: a negative pressure conduit (e.g., a tube coupled to a vacuum pump to generate a vacuum over a treatment area); a positive pressure conduit (e.g., a tube coupled to an air pump to allow the device to be released after a treatment and/or to “float” over the skin as the device is moved into a position over the skin); and an object to mechanically push the skin (e.g., a piston or plunger to push blood away from a treatment area just before exposing the area to energy); and multiple, different sources of energy (e.g., several light sources of different wavelengths or other properties); and one or more sensors (e.g., one or more skin color sensors or skin temperature sensors to provide feedback to a user, or to an automatically controlled processing system before, during, or after a treatment; and a pixilated display having rows and columns of pixels on a portion of the device (e.g., a backlit liquid crystal display device which displays skin temperature and other information); and two different vacuum regions, a first vacuum region creating a vacuum in a border region of external biological tissue which surrounds a desired treatment area of external biological tissue and a second vacuum region which applies a vacuum to the desired treatment area after a vacuum has been applied to the border region; and other aspects and/or features described herein.

Various methods of operating these apparatuses are also described. One exemplary method for treating a target with a device includes applying the device to an area of biological external tissue having a target, applying a negative pressure (e.g., a vacuum) on the area, then applying an energy (e.g., laser light) to the area under negative pressure, and after applying the energy, applying a positive pressure to the area to allow the device (e.g., a handheld device) to be easily released from the treatment. The positive pressure may be a cooling gas. Other exemplary methods are also described.

### BRIEF DESCRIPTION OF THE DRAWINGS

The present invention is illustrated by way of example and not limitation in the figures of the accompanying drawings in which like references indicate similar elements.

FIG. 1a is a diagram showing the various layers of the skin and potential targets for photo therapy and/or electrical therapy.

FIG. 1b shows the percentage of incident energy transmitted through the epidermis for three different skin types.

FIG. 1c shows the percentage of incident energy transmitted through the dermal plexus for two different levels of blood concentration (shown as ratios of blood to the rest of the tissue in a given volume).

FIG. 2a is a process flow diagram showing a method of applying positive pressure and negative pressure to biological external tissue having a target.

FIG. 2b is a process flow diagram showing a method for applying negative pressure to biological external tissue having a target.

FIG. 2c is a process flow diagram showing a method for applying a sequence of positive pressure, negative pressure, and positive pressure to biological external tissue having a target.

FIG. 3 shows, in cross sectional view, a device 300 having multiple light sources 303a, 303b, and 303c, and a pressure conduit 304.

FIG. 4 shows, in cross sectional view, a device 400 having a pair of electrodes 403a and 403b, an object 401, a pressure conduit 404 and an electric current passing through biological external tissue 302.

FIG. 5 shows, in cross sectional view, a device 500 having multiple energy sources 503a-c, an object 401 and a pressure conduit 504.

FIG. 6 shows, in cross sectional view, a device 600 having multiple energy sources 503a-c, a pressure conduit 504, and a skin temperature sensor 601.

FIG. 7 shows, in cross sectional view, a device 700 having multiple energy sources 503a-c, a pressure conduit 504, a membrane 301, electrodes 503d and 503e, and a skin color sensor 701.

FIG. 8 shows an exemplary display 800 on a handheld device according to certain embodiments of the invention.

FIG. 9 shows a handheld device 900 with a display element 901 that displays at least one parameter with respect to a treatment of the biological external tissue 302.

FIG. 10 shows a device 1000 having multiple energy sources 503a-503e that are not exposed to any pressure, and a pressure conduit 1004.

FIG. 11 shows a device 1100 having a body that is applied to biological external tissue 302 and multiple vacuum chambers as shown in A and B on FIG. 11.

FIG. 12 shows a device that is an apparatus 1200 that attaches to an existing device 1201 to apply energy to biological external tissue 302 through energy sources 503a-c.

FIG. 13 shows an electrical schematic of a handheld device according to one exemplary embodiment.

### DETAILED DESCRIPTION

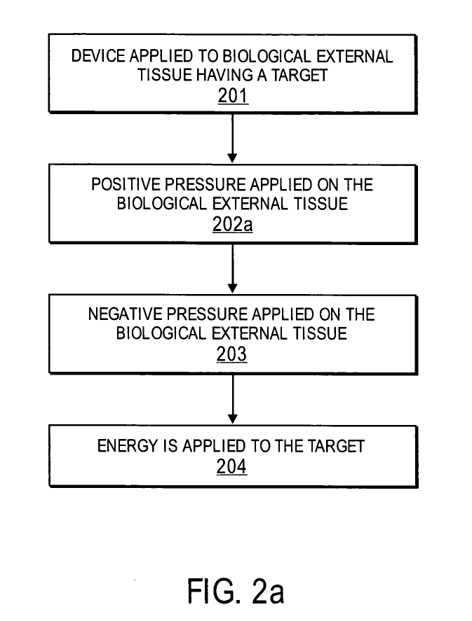

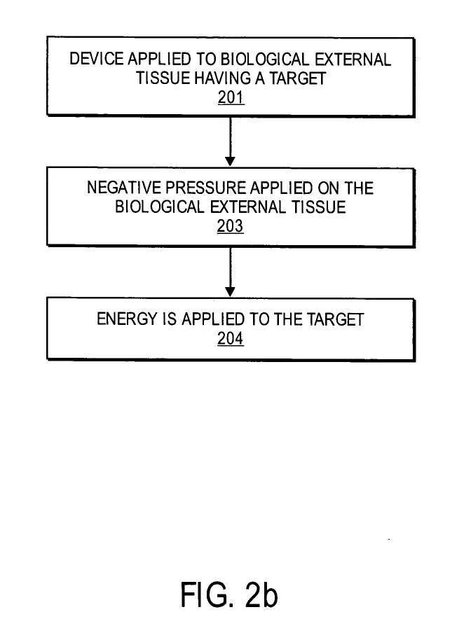

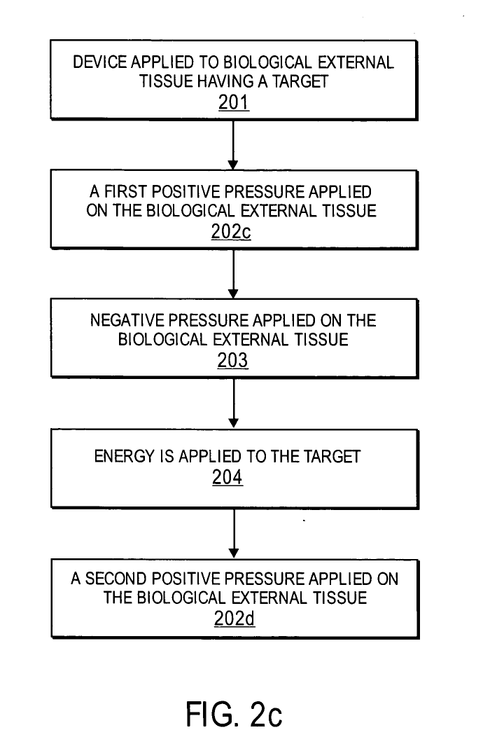

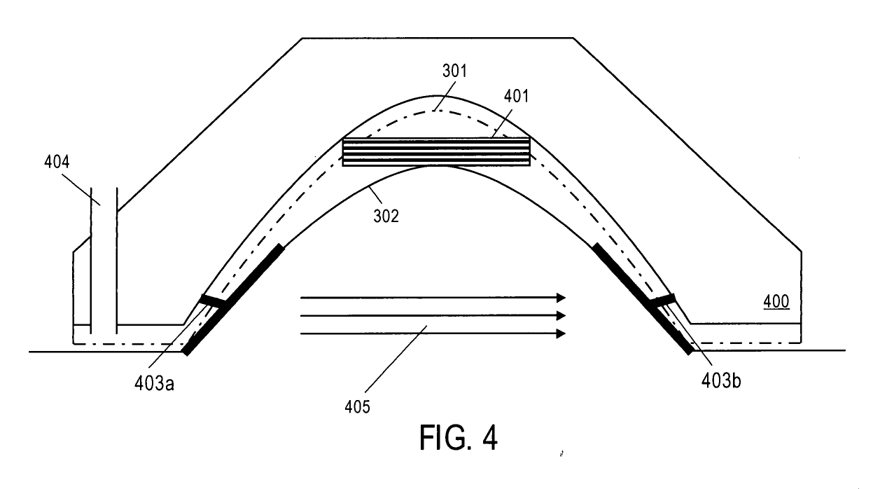

Prior to describing specific devices which are embodiments of the invention, several methods which are also embodiments of the invention will be described. FIG. 2a is a process flow diagram showing a method of applying positive pressure and negative pressure to biological external tissue having a target. According to one embodiment of the invention, when the negative pressure is applied to the skin and the volume of biological external tissue is pulled into the device, blood is pulled into the dermal plexus and the dermis. In operation 201 a device is applied to biological external tissue having a target. The device may be, for example, the device 400 shown in FIG. 4. According to one embodiment of the invention, the biological external tissue is dermalogical tissue and the device is applied by pressing the device against such tissue to create a sealed region between the device and such tissue. The target is skin lesions in one embodiment of the invention. In another embodiment of the invention, the target is melanin, blood, tattoo ink, and/or collagen. However, the invention is not so limited. The target can alternatively be any biological external tissue requiring treatment by an energy source. In operation 202a a positive pressure is applied to the biological external tissue.

According to one embodiment of the invention, the positive pressure is applied using an object which protrudes from a surface of a body of the device (such as object 401) which surface faces the area to be treated. According to another embodiment of the invention, the positive pressure is a gas such as a cooling gas, which is applied to the biological external tissue. In operation 203 of FIG. 2a, a negative pressure is applied to the biological external tissue. According to one embodiment of the invention, the negative pressure is a vacuum (e.g., a pressure which is less than or substantially less than atmospheric pressure, such as 400 torr). In operation 204, energy is applied to the target inside the biological external tissue. The energy is incoherent light, coherent light, radio frequency, or ultrasound, according to various embodiments of the invention. However, the invention is not so limited. The energy source may be a combination of multiple energies such as a radio frequency and a coherent light in some embodiments of the invention. In another embodiment of this invention, pressurized gas is used to force the blood out of the dermal plexus. The positive pressure applied in operation 202a tends to push blood out of the treatment area, thereby reducing the amount of energy absorption by the blood in the treatment area. This pushing of blood normally occurs just before the application of energy to the treatment area.

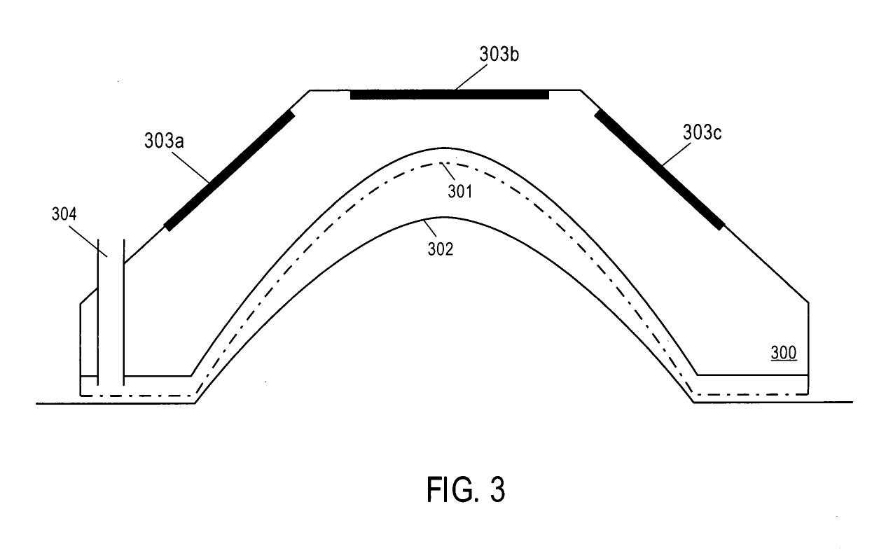

FIG. 2b is a process flow diagram showing a method for applying negative pressure to biological external tissue having a target. In operation 201 of FIG. 2b, a device (such as, for example, the device 300 shown in FIG. 3) is applied to biological external tissue having a target; operation 201 of FIG. 2b may be similar to operation 201 of FIG. 2a. In operation 203 of FIG. 2b, a negative pressure is applied to the biological external tissue. In operation 204 of FIG. 2b, energy is applied to the target, which may be energy as described with reference to FIG. 2a. In FIG. 2b, no positive pressure is applied to the biological external tissue prior to the negative pressure being applied.

FIG. 2c is a process flow diagram showing a method for applying a sequence of positive pressure, negative pressure, and positive pressure to biological external tissue having a target. In operation 201 of FIG. 2c, a device (such as, for example, the device 400 shown in FIG. 4) is applied to biological external tissue having a target, as described with reference to FIG. 2a. In operation 202c, a first positive pressure is applied to the biological external tissue. As described with reference to the method of FIG. 2a, the positive pressure may be a cooling gas or an object. In operation 203 of FIG. 2c, a negative pressure is applied to the biological external tissue; this is simlar to operation 203 of FIG. 2a. In operation 204 of FIG. 2c, energy is applied to the target; this is similar to operation 204 of FIG. 2a. In operation 202d, a second positive pressure is applied on the biological external tissue. This second positive pressure may be a gas which pushes the device off the biological external tissue, thereby making it easier to release and move the device from the treatment area to the next treatment area. According to some embodiments of the invention, the first positive pressure and the second positive pressure originate from the same pressure source. In some embodiments of the method of FIG. 2c, operation 202c may overlap in time with operation 203 or the sequence may be reversed. Normally, the negative pressure is applied while the energy is applied so operations 203 and 204 overlap substantially in time.

In alternate embodiments of the invention, the first positive pressure and the second positive pressure are different positively applied pressures on the biological external tissue. For example, the first positive pressure is applied by a mechanical object (e.g., object 401) while the second positive pressure is applied by pumping a gas (e.g., air) into the recess between the device and the skin or other biological external tissue. In some embodiments of the process flows of the invention, as shown in FIGS. 2a, 2b and 2c, the number of uses of the device is kept track of to determine usage patterns of the device. The energy used in the methods of FIGS. 2a, 2b, and 2c, may originate from a source that is not exposed to any negative or positive pressure according to at least one embodiment of the invention. In another embodiment of the invention, generating a peripheral vacuum seal to keep the device on the area of biological external tissue can also be performed and is described further below.

The energy may be an electrical current that is applied to the area of biological external tissue before the blood concentration in the area returns to a normal state (or higher than normal state), according to some embodiments of the invention. Furthermore, measuring color of the biological external tissue can alternatively be performed in some embodiments of the methods shown in FIGS. 2a, 2b and 2c. Similarly, measuring temperature of the biological external tissue may also be performed in some embodiments of the methods shown in FIG. 2a, 2b and 2c. The device may display at least one measurement of a sensor on the device in some embodiments of the invention. According to one embodiment of the invention, temperature can be measured by monitoring the change in electrical impedance of the treatment volume. The device may be a handheld device in some embodiments of the invention. In other embodiments, a power source may provide power to the device and generate the positive pressure and/or negative pressure through a pressure source connected to the device through a cable element.

In some embodiments of the invention, the strength of the energy may be automatically regulated by a controller. The controller may also perform other functions. The controller may, for example, contain a timer that is monitoring the elapsed time since a positive pressure is applied to the treatment volume, according to one embodiment of the invention. The result of a large elapsed time is a pool of blood that returns to the surface of biological external tissue such as skin. All skin types including type VI assume a more reddish appearance. The presence of this pool of blood significantly impacts the therapy. The blood absorbs much of the light energy particularly if the energy is in the visible portion of the spectrum. If the target such as a hair follicle, a tattoo, or collagen is deeper in the body than the pool of blood, the therapy is unsuccessful as the majority of the treatment energy is absorbed in the pool of blood before reaching the intended target.

Based upon clinical measurements, the blood volume in the dermal plexus and dermis is reduced for a period time before it refills the capillaries and other vessels in these regions. This period of time is on the order of 100 msec, but varies from individual to individual. By monitoring the elapsed time since application of a positive pressure, the treatment (e.g., application of energy) can be performed in this time period before the blood refills this tissue.

After the controller determines the tissue is in place and, if required, the elapsed time is less than the blood refill time, the therapy is applied to the volume of skin contained inside the device. If photo-therapy is used, an intense light such as from a laser or a flash lamp is directed onto the treatment area of the biological external tissue. If rf therapy is used, an electrical voltage is applied to the electrodes and current is passed through the volume of tissue between the electrodes. Once the therapy is completed, the negative pressure is removed and the skin returns to its normal state.

A controller may function in the following manner in the case of a device 400 of FIG. 4. This particular device 400 may provide a positive pressure whenever it is being moved from one treatment area to another treatment area. As noted above, the device typically has a recessed area which faces the skin and which is enclosed by the device and the skin when the device is pressed against the skin. The positive pressure (e.g., from a gas) is typically emitted from the recessed area, and this positive pressure will cause a pressure buildup when the device is pressed against the skin to create a seal between the device and the skin. When the device is being moved, there is no seal and thus no pressure buildup between the skin and the device. When it is pressed against the skin, the positive pressure (e.g., a pressure greater than atmospheric pressure) between the device and the skin will be measured by a pressure sensor, and this indicates to the controller that the movement of the device has stopped and that the user has positioned the device over a desired treatment area. At this point, the controller may be programmed as built to automatically shut off the positive pressure and begin drawing a vacuum against the skin to lock the device in place over the desired treatment area. Alternatively, the controller may be programmed or built to merely stop the positive pressure (e.g., shut off the flow of a gas into the recess which creates the positive pressure) but not start a vacuum until the user of the device switches a vacuum on. This alternative implementation gives the user a chance to adjust the positioning before turning the vacuum on by a command from the user.

The biological external tissue that is outside of the device may be prevented from stretching in some embodiments of the methods shown in FIGS. 2a, 2b and 2c. A technique for preventing this stretching is described below.

FIG. 3 shows, in cross sectional view, a device 300 having multiple light sources 303a, 303b, and 303c, and a pressure conduit 304. The light sources are contained within a housing or body which also includes a cover (which is transparent in the case of light sources) and which separates the light sources from any vacuum generated between the skin and the device). The cover is disposed between the membrane 301 and the light sources 303a-303c. A handle which is coupled to the body may also be included so that a user of the device can easily hold and move the device over a patient's skin or other biological external tissue.

A recess or void exists between the membrane 301, which faces the biological external tissue 302, and the biological external tissue 302 shown in FIG. 3. Pressure conduit 304 generates a negative vacuum through membrane 301 to bring the biological external tissue 302 into the recess and toward the membrane 301. Membrane 301 can be used to collect dead skin, according to one embodiment of the invention. The membrane 301 is coupled to the conduit 304 to receive the suction from a vacuum pump (not shown) which is coupled to the conduit 304. Light sources 303a, 303b and 303c in FIG. 3 are connected to an energy source that is not shown on the figure, according to one embodiment of the invention. This energy source is not exposed to any pressure through pressure conduit 304, according to one embodiment of the invention. These light sources are shielded from any negative (or positive) pressure by the cover which is optically transparent in the case where the energy sources provide visible light. It will be appreciated that the light sources may alternatively be other types of energy sources (e.g., microwave radio frequency energy) which may not require an optically transparent cover.

The energy applied to biological external tissue 302 through device 300 is transferred through light sources 303a, 303b and 303c. The light sources 303a, 303b, and 303c may include, for example, light emitting diode (LED) lasers of different wavelengths, thus providing different energy sources, due to the different wavelengths, in the body of the device. Each light source (e.g., source 303a or 303b or 303c) may be a panel of multiple LED lasers which may be the same type of LED (to produce the same wavelength) or may be a panel of multiple LED lasers which may be a different type of LED (to produce different wavelengths). The three panels shown in FIG. 3 (light sources 303a, 303b, and 303c) are arranged within the body of device 300 to provide a spatially uniform lighting at the target so that the intensity of light, at any point over an area which includes the target, is substantially the same. It can be seen from FIG. 3 that the panels (e.g., light source 303a) transmit light directly to the target without any intervening optical fibers or waveguides.

This energy for device 300 can be incoherent light, coherent light, or alternatively non-visible light or electromagnetic radiation in the range of a radio frequency spectrum, or ultrasound, according to various embodiments of the invention. The energy source for the device 300 may be a flash lamp, arc lamp, high frequency electrical energy, rf energy, an LED or a Direct Current electrical energy, according to various embodiments of the invention. However, the invention is not so limited. The present invention can be multiple combinations of different energies which are provided by energy sources in the body of device 300. The device 300 may also be connected to a pressure source in the device 300 for providing power to device 300 and generating pressure through pressure conduit 304 in one embodiment of the invention. In another embodiment of the invention, the device 300 may be a handheld device that is connected to the pressure source (through a cable element), where the pressure source and power source is separate from the handheld device. In addition, a controller on or near device 300 may control the strength of the energy applied through light source 303a, 303b or 303c. According to one embodiment of the invention, there are three light sources, however, any number of light sources is contemplated by the present invention. In one embodiment of the invention, a tapered outer wall on the periphery of device 300 prevents the biological external tissue 302 that is outside the device 300 from stretching.

Stretching the skin (1) reduces the concentration of melanin in the epidermis, (2) reduces scattering in both the epidermis and the dermis, and (3) moves the treatment target closer to the surface. Vacuum provides an excellent mechanism for stretching the skin. By sealing on an area of skin, and generating a vacuum, the skin is drawn and stretched much more than can be done manually.

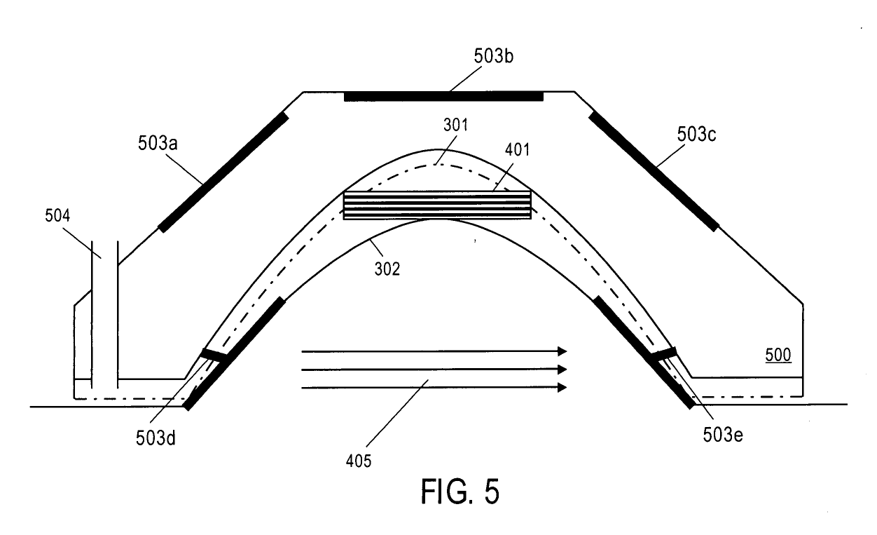

FIG. 4 shows, in cross sectional view, a device 400 having a body which is coupled to a pair of electrodes 403a and 403b, and the body supports an object 401 which protrudes into a recess of the body. A pressure conduit 404, which is coupled to the body, generates a positive or negative pressure on biological external tissue 302. The object 401 is designed to be brought into contact with biological external tissue 302 either before or while a negative pressure through pressure conduit 404 is applied, thereby drawing the skin into the recess and into contact with the object. The object is used for pressing onto the biological external tissue 302 and forcing the blood out of the dermal plexus, according to one embodiment of the invention. The object 401 may be stationary relative to the body or it may move, like a plunger or piston, down from the body and toward the skin. A stationary object is simpler and easier to build but will require that the vacuum draw the skin sufficiently into contact with the object. The moving object can provide more force and the recess can be larger. The object 401 may be transparent in the optically visible spectrum, thereby allowing light to pass through it in those embodiments (such as, e.g., the device of FIG. 5) which include light sources which emit light which must pass through the object to reach the target.

According to some embodiments of the invention, pressure conduit 404 generates a positive pressure that is a gas, which may be a cooling gas. According to one embodiment of the invention, the gas that is used to apply pressure to the biological external tissue 302 to force the blood out of the dermal plexus and the dermis may also be used to assist in releasing the device 400 from the biological external tissue 302. In another embodiment of the invention, the cooling gas is applied before applying an electric current 405 through the biological external tissue 302 through electrodes 403a and 403b. In another embodiment of the invention, the pressure conduit 404 generates a peripheral vacuum seal to hold device 400 on biological external tissue prior to generating a vacuum in the recess of the body.

The object 401 that applies pressure to the biological external tissue 302 to force the blood out of the dermal plexus and the dermis may be cooled to a temperature lower than the epidermis, according to one embodiment of the invention. Without cooling, the normal epidermis starts at a temperature between 31 and 33C, according to one embodiment of the invention. During treatment, it will rise in temperature and may reach a temperature at which burning occurs. If the epidermis starts at a temperature lower than normal, it can change in temperature during treatment more than uncooled skin before it reaches a temperature at which burning occurs.

The gas that is used to apply pressure to the biological external tissue 302 to force the blood out of the dermal plexus and the dermis may be cooled to a temperature lower than the epidermis, according to one embodiment of the invention. The benefit of this cooling with pressurized gas is the same as the benefit obtained with a cool object 401. The object 401 that applies pressure to the biological external tissue 302 to force the blood out of the dermal plexus and the dermis may contain an optical coating to control the wavelengths of light that are used in the treatment, according to another embodiment of the invention. In some embodiments of the invention, the object 401 that applies pressure to the skin to force the blood out of the dermal plexus and the dermis may contain an optical coating to control the energy of the light that is used in the treatment. According to one embodiment of the invention, DC or AC or capacitance electrical sensors 403a and 403b are used to determine if the biological external tissue 302 is properly positioned in the device 400.

The device as shown in FIG. 4 can include various sensors such as skin color sensors, temperature sensors, and capacitance sensors on the device in some embodiments of the invention. Furthermore, the device shown in FIG. 4 may have a tapered outer wall on the periphery of the device that prevents the biological external tissue 302 that is outside of the device 400 from stretching, similarly to as described with reference to FIG. 3. Other features from other embodiments described herein may also be added to the device as shown in FIG. 4.

The electrodes 403a and 403b in FIG. 4 can serve two purposes. One purpose is for applying rf treatment energy according to one embodiment of the invention. The second purpose is as an electrical sensor, according to a different embodiment of the invention. An AC or DC voltage is applied to at least two of the electrical sensors in other embodiments of the invention. When the biological external tissue 302 contacts two of the electrical sensors 403a and 403b, an electrical current 405 passes between the two electrodes 403a and 403b. When a sensor within device 400 detects this current 405, it signals a controller within or outside device 400. The controller interprets this signal to mean that the biological external tissue 302 is properly positioned according to one embodiment of the invention. This can serve as a secondary skin detection system for added safety, according to at least one embodiment of the invention.

FIG. 5 shows in cross sectional view, a device 500 having multiple energy sources 503a-c, an object 401 and a pressure conduit 504. In a typical treatment, the device 500 is pressed against the skin, and the skin is drawn into the recess of the body of device 500 as shown in FIG. 5. According to one embodiment of the invention, the device 500 generates a positive pressure against the skin (through the object 401) followed by a negative pressure (through a vacuum pump coupled through a valve to conduit 504), and then again a positive pressure (from an air pump coupled, through a valve, to conduit 504) to be applied to biological external tissue 302 through pressure conduit 504. The positive pressure from the object 401 may be done concurrently with the generation of a vacuum (negative pressure) in the recess. This sequence helps certain treatment procedures of biological external tissue 302 requiring blood within the biological external tissue 302 to be pushed away prior to the treatment. FIG. 5 differs from FIG. 3 and FIG. 4 in that the device shown in FIG. 5 can generate both an electric current through electrodes 503d and 503e (to either sense the device's contact with the skin or to deliver electrical energy as a treatment) and can apply energy through sources 503a, 503b and 503c on device 500. The energy sources 503a, 503b, and 503c may be similar to the sources 303a, 303b, and 303c. However, the energy through energy sensors 503a, 503b and 503c is not limited to light, according to one embodiment of the invention as shown in FIG. 5. The pressure conduit 504 generates at one point in time in a treatment sequence, a positive pressure comprising a gas in an area of the biological external tissue 302 in FIG. 5. However, the pressure conduit 504 can alternatively generate negative pressure at a different time in the sequence by switching a valve which connects the conduit to either an air pump or a vacuum pump. Other features (such as, e.g., skin color sensors, a display, etc.) from other embodiments described herein may also be implemented on the device as shown in FIG. 5.

In FIG. 5, a high frequency rf electrical current 405 enters the body from one electrode 503d, passes through a layer of biological external tissue 302 and exits the body at a different electrode 503e. FIG. 5 shows a potential pathway through the biological external tissue 302 for this current 405. As the current 405 passes through the body, it tracks a path through the least resistive tissues. Blood is the most conductive biological entity and hence the rf electricity tends to track the blood vessels. This is fine if the target for the rf is the blood, but if the target is the adjacent tissue such as collagen, the presence of the blood can defeat the intended therapy.

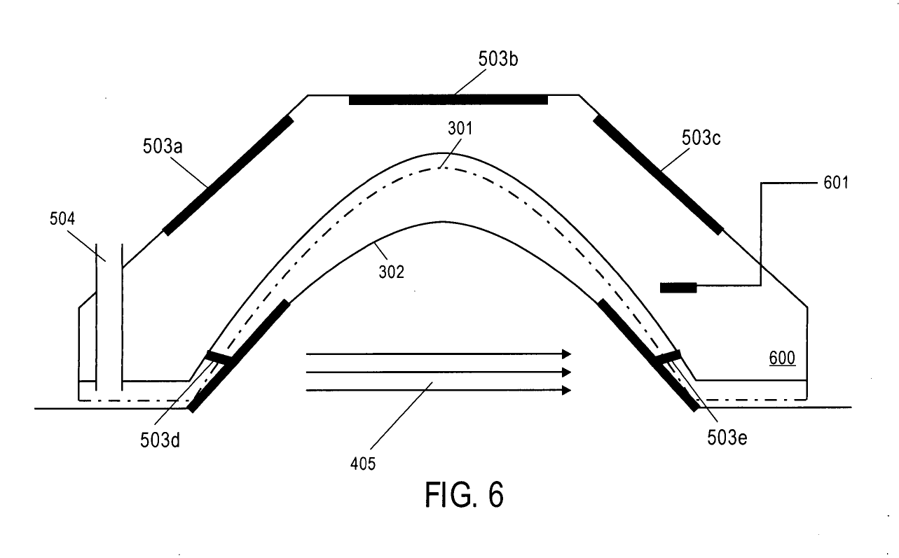

FIG. 6 shows in cross sectional view, a device 600 having multiple energy sources 503a-c, a pressure conduit 504, and a skin temperature sensor 601. The skin temperature sensor 601, as shown in FIG. 6, is a capacitance sensor. It may be placed on the membrane 301 rather than within the body of the device. In one alternative embodiment of the device 600, an object 401 may also be used, as shown with reference to FIG. 4. Furthermore, other features from other embodiments described herein may be added to the device 600 shown in FIG. 6. The skin temperature sensor 601, as shown on device 600 in FIG. 6, is used to measure the temperature of the biological external tissue 302 to prevent burning when applying energy through one or more of energy sources 503a-c to biological external tissue 302.

According to one embodiment, the skin temperature sensor 601 is a non-contact skin temperature sensor that monitors the infrared light emitted from the surface of the biological external tissue 302 and translates this into a surface temperature. The information from the skin temperature sensor 601 is sent to a controller which is within the body of device 600 in certain embodiments of the invention. The controller is a micro controller or microprocessor that interprets the skin temperature, and if the temperature has reached a dangerous level, the micro controller terminates the application of energy in one embodiment of the invention According to another embodiment of the invention, the controller is a software controlled micro controller or microprocessor.

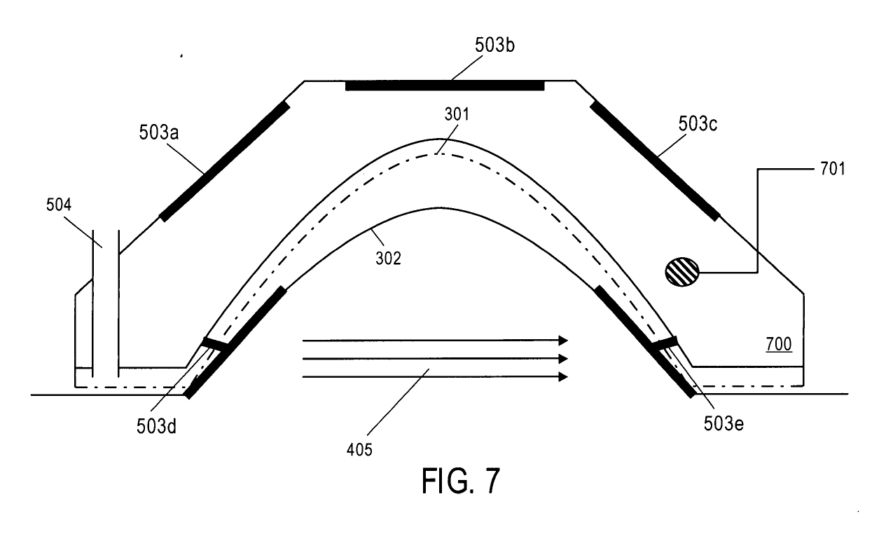

FIG. 7 shows in cross sectional view, a device 700 having multiple energy sources 503a-c, a pressure conduit 504, a membrane 301, electrodes 503d and 503e, and a skin color sensor 701. FIG. 7 differs from FIG. 6 in that it does not have a skin temperature sensor 601, but rather has a skin color sensor 701. The skin color sensor 701 is used to measure the level of energy that needs to be applied to biological external tissue 302 based upon the color of the skin and corresponding melanin and blood levels within biological external tissue 302. Other features (such as, e.g., an object 401, etc.) from other embodiments described herein may be added to the device shown in FIG. 7.

The skin color sensor 701 consists of a light source and a photodiode. By shining the light source on the surface of the biological external tissue 302 and reading its reflection with the photodiode, the skin color can be determined. The light source may be adjacent to the photodiode (as shown), or it may be separated from it. Determining the skin color prior to treatment is important. Even with stretching, dark skin is still more susceptible to burning than lighter skin. Consequently the treatment energy may be adjusted based upon the readings of the skin color sensor. For darker skin, the treatment energy is lowered. For lighter skin, the treatment energy is raised.

Clinical tests of device 700 on lighter skin types shows that the skin color sensor (4) can also be used to detect the absence of the blood and further detect the refill of the vessels in the dermal plexus and dermis. Prior to stretching the biological external tissue 302, such as skin, into the device 700, the skin color is measured. As the skin is stretched and the blood is removed from the dermal plexus, the reflected light detected by the photo diode increases due to less absorption by the blood. As the dermal plexus refills, the reflected signal decreases due to increase absorption by the blood. The skin color detection device monitors this change and notifies a control system within or outside device 700, according to certain embodiments of the invention.

Stretching the epidermis reduces the concentration of melanin. To understand this phenomenon, consider a colored balloon. The pigmentation in the balloon gives it its color. The melanin pigmentation in our skin gives us our color. When a colored balloon is deflated, it is difficult or impossible to see through it. It is opaque. As the balloon is inflated, it becomes more transparent. The elastic portion of the balloon stretches. The inelastic portion, such as the pigment, does not stretch. Its concentration is reduced and the balloon becomes more transparent. The same happens in our skin. The melanin is less elastic that the interstitial components. These tissues stretch while the melanin does not. As the concentration of melanin drops, the skin becomes whiter. In fact, by stretching the skin of a dark individual, the skin becomes quite pink as the underlying vascular system becomes exposed.

The second advantage of stretching the skin prior to and during treatment with intense light sources is the reduction in scattering. When light enters human tissue, it is immediately scattered in all directions by the collagen, fibrous tissue and other intercellular constituents. Much of this light is scattered back to the surface and out of the body. Much is scattered sideways and thereby reduces the energy density as the cross section of the intense light source increases. The level of scattering is directly proportional to the concentration and orientation of the intercellular material. Stretching the skin reduces the concentration of these materials in direct proportion to the level of stretching. The corresponding scattering is subsequently reduced as well.

As described above, the two advantages to stretching the skin is reduced absorption by melanin and reduced scattering. The third advantage is the treatment target moves closer to the surface. Stretching the skin reduces its thickness. One can see this by taking a rubber band and measuring its thickness. Then stretch the rubber band and measure its thickness a second time. The rubber band is thinner. The same effect occurs with the outer layers of the skin. The epidermis becomes thinner. The dermal plexus becomes thinner. Even the dermis becomes thinner. The target however, remains in the dermis and is now closer to the surface and thus more energy can reach it.

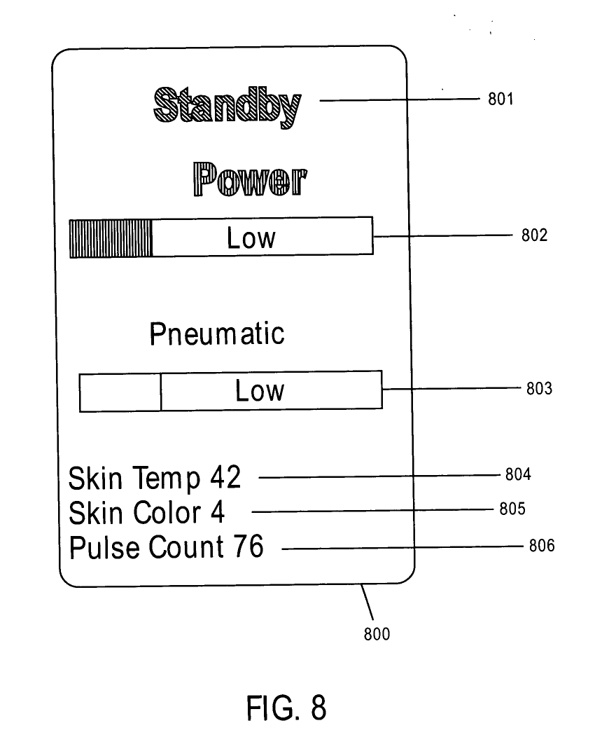

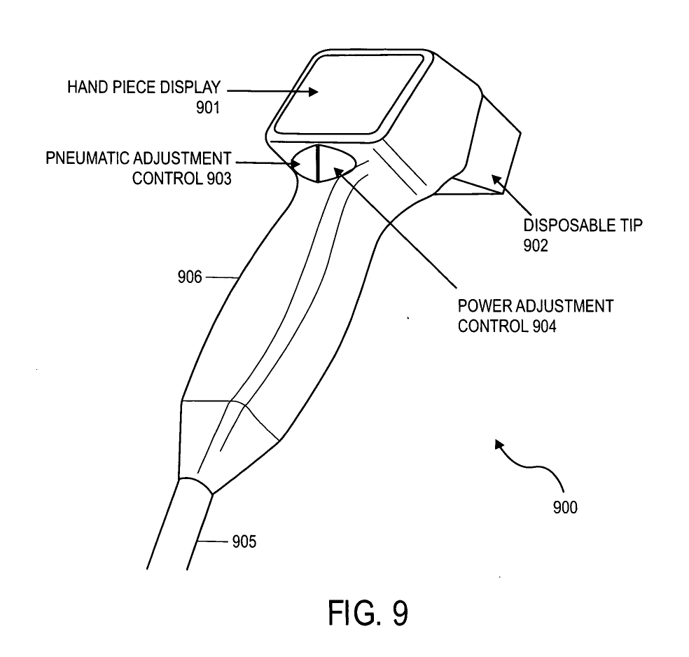

FIG. 8 shows an exemplary display which may be disposed on a surface of a handheld device, such as any of the devices shown in FIGS. 3-7 and 9-11. FIG. 9 shows a perspective view of a handheld device 900 with a display on a surface of the device. The device of FIG. 9 may include the various features described herein, such as multiple energy sources, an object which pushes blood out of the treatment area, one or more pressure conduits, etc. The device 900 includes a pixilated display with multiple rows and columns of pixels on the display 901. An example of the content of such a display is shown in FIG. 8 which shows a display 800 which indicates the status 801 of the device (e.g., “Standby” or “On” or “Treating”), the power status 802 of the device (e.g., Low or Medium or High along with a bar graph which indicates the power status), the vacuum status 803 of the device (e.g., pneumatic level is “Low” or “High”), the skin's temperature 804 (e.g., 42° C.), the skin's color 805 (e.g., 4) and the patient's pulse count 806 (e.g., 76). The display 800, being on the handheld, is easier for an operator (e.g., physician) to see while doing a treatment because the operator can look at the treatment site while operating the device and still be able to see both the site and the display (rather than having to look at a console which has a display and which is separate from the handheld device. The display 901 may be a liquid crystal display (LCD) or an LED display which is controlled by a display controller which updates the display's pixels to reflect new information. The device 900 includes a power adjustment control 904 which can be used to control the amount of energy that is applied to the biological external tissue (e.g., to adjusting the intensity of the light from light sources). The device 900 also includes a pneumatic adjustment control 903 to control the strength of a vacuum that is applied through a vacuum pump (not shown) through the device 900 (e.g., (e.g., a pressure which is less than or substantially less than atmospheric pressure, such as 400 torr). Furthermore, the device 900 includes a cable 905 that delivers power and pressures to operate device 900 (e.g., the cable 905 is connected on the other end to a wall power outlet, or a standalone central control station); a vacuum through device 900 to be applied the biological external tissue in front of the disposable tip 902 (e.g., the vacuum may be delivered through conduit 905 along with power by maintaining a separate chamber that separately carries a negative pressure through device 900); a positive pressure to press down on biological external tissue (e.g., carried through a separate chamber than the one that carries the vacuum and power); and the cable 905 may optionally include various electrical wires that deliver signals to and from various sensors (e.g., sensors on the device 900 may include skin temperature sensors, skin color sensors, and capacitance sensors, etc.) on device 900 to a standalone central control station (not shown) in addition to (or rather than) the hand piece display 901. In one embodiment, the standalone central control station may be a computer that has a printer and/or storage device(s) for recording data from the sensors on device 900. The disposable tip 902 on device 900 may be a disposable membrane 301 or may be custom designed to fit a particular type of biological external tissue or size of biological external tissue (e.g., the disposable tip 902 may be different for large areas of skin verses small areas of skin, and may be shaped differently to treat areas of biological external tissue that is not purely flat because of contours created by skeletal structures and/or because of hair follicles). The handle 906 of device 900 may be designed to fit a particular size of hand or may have groves to fit a particular hand size in some embodiments. In addition, in other embodiments the handle 906 may be of variable size (e.g., to fit larger and smaller hands, or to reach into areas of biological external tissue that are otherwise difficult to reach). The handle 906 may be removable from the device 900 head (e.g., the head might be the handpiece display 906 and disposable tip 902 together) in one embodiment to allow a user of device 900 to quickly put on different types of sensors, display 901 variations, and disposable tip elements 902.

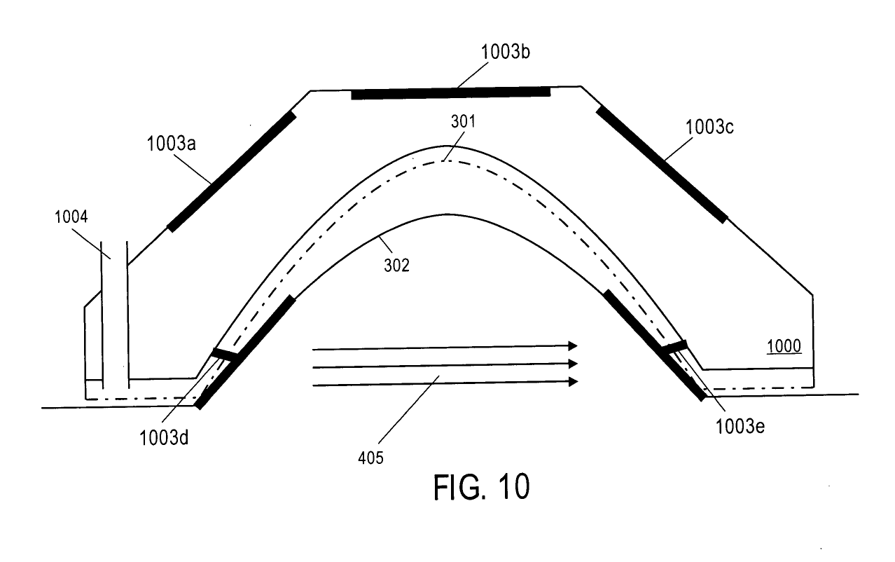

FIG. 10 shows a device 1000 having multiple energy sources 503a-503e that are not exposed to any pressure, and a pressure conduit 1004. FIG. 10 differs from FIG. 3 in that the device shown in FIG. 10 includes multiple energy sources such as electrodes 1003d and 1003e, while the device shown in FIG. 3 is limited to light based energy only. In one embodiment of the present invention, the pressure conduit 1004 in FIG. 10 generates a negative pressure.

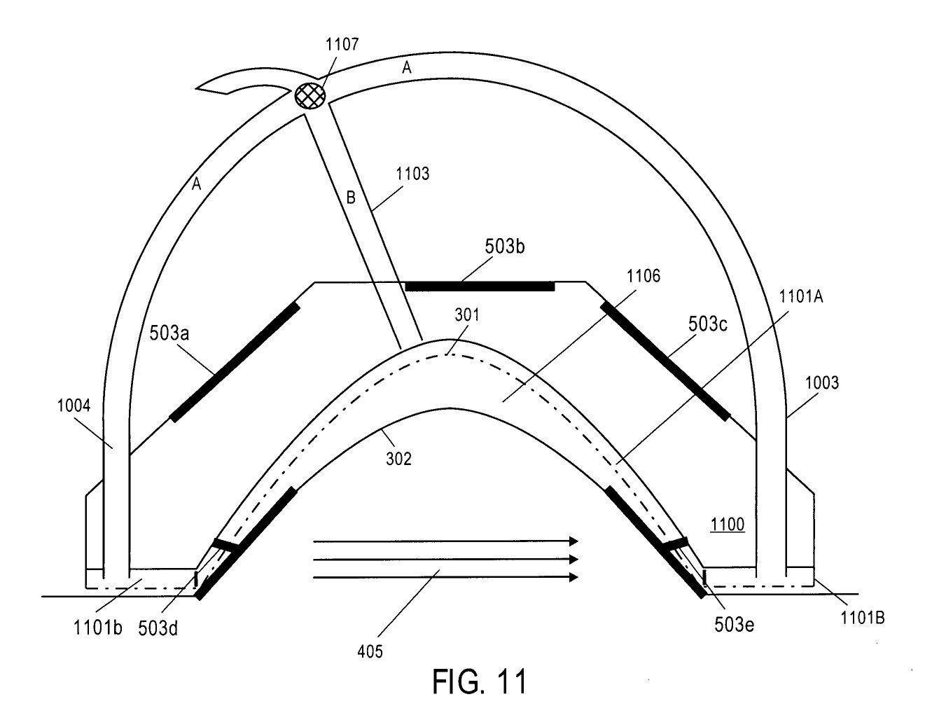

FIG. 11 shows a device 1100 having a body that is applied to biological external tissue 302 and multiple vacuum chambers shown as A and B on FIG. 11. The device 1100 in FIG. 11 applies two vacuum pressures at different times to biological external tissue 302. In other embodiments of the invention as shown in FIG. 11, there are any number of vacuum chambers A, B on device 1100. One pressure A is generated at the periphery of device 1100 through the pressure conduits 1004 and 1003. A second pressure is generated as shown in B through pressure conduit 1103. The device 1100 includes multiple energy sources 503a, 503b, and 503c and electrodes 503d and 503e. The membrane 301 has two portions: an interior portion 1101A which generates an interior vacuum in the recess 1106 of the body of device 1100 and a peripheral border portion 1101B which generates a peripheral vacuum seal between the flat surface of the periphery of the device 1100 and the skin. A valve 1107 couples the two vacuum chambers together an it may be manually controlled by an operator or automatically controlled by a micro controller (e.g., micro controller 1303 in the handheld device). Initially, the valve 1107 is set so that a vacuum is generated in only the peripheral border of the device; the peripheral border may be a rectangular frame (resembling a picture frame) or other shapes. This clamps the device to the skin without creating a vacuum in the recess 1106. Then the valve 1107 is switched so that a vacuum is generated in both the peripheral border and the recess 1106 of the device. In an alternative embodiment, the valve may be positioned at the junction between the portion 1101A and 1101B and no separate conduit 1103 is required; in this case the valve is switched open to extend a vacuum from the peripheral border region to the interior region. The advantage provided by a device such as device 1100 is that the skin within the recess can be stretched even more than skin within devices such as device 300 or 400 because less skin outside of device 1100 will be pulled in by the vacuum within the recess. The skin in the peripheral border region is clamped into a relatively fixed position before the skin within the recess is exposed to a vacuum, and this tends to prevent skin from being pulled into device 1100 from outside of the device 1100. One or more features (such as, e.g., an object 401, skin color sensors, pressure sensors, a display on the handheld, etc.) from other embodiments described herein may be added to the device 1100 according to certain implementations of the invention.

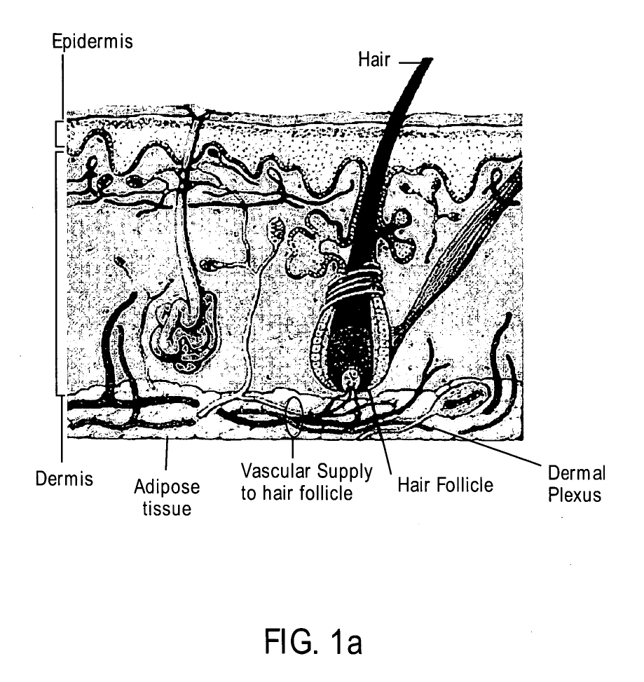

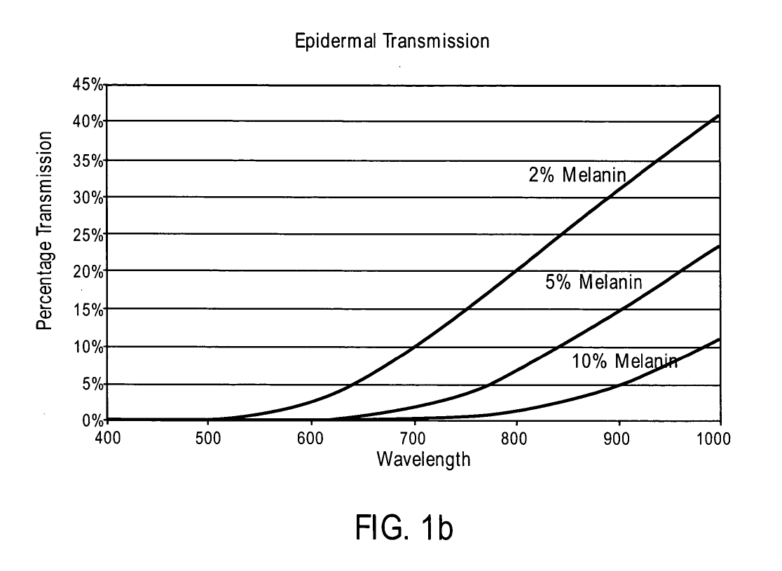

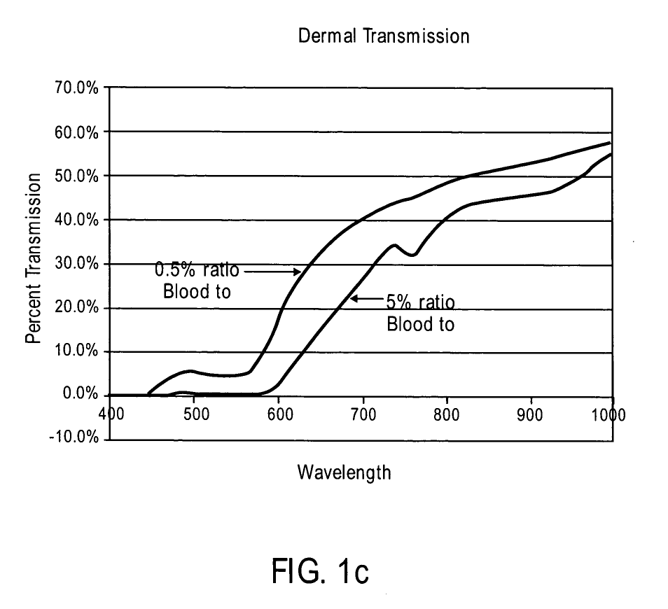

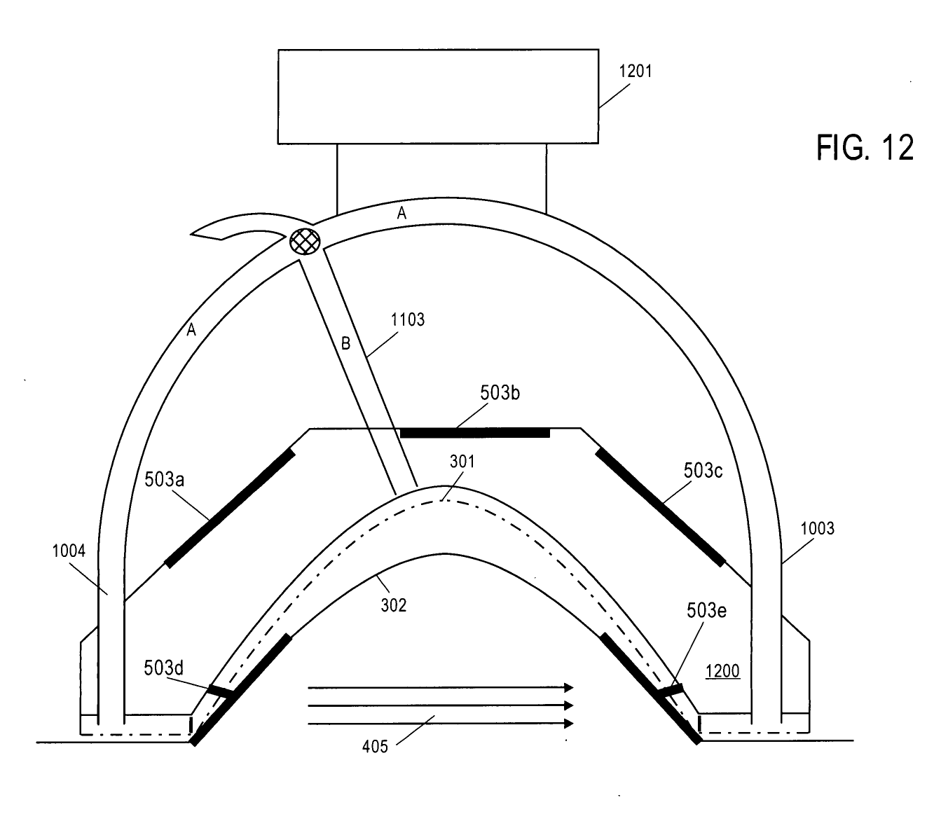

FIG. 12 shows a device that is an apparatus 1200 that attaches to an existing device 1201 to apply energy to biological external tissue 302 through energy sources 503a-c. The apparatus shown in FIG. 12 is an embodiment of the invention that is an add-on to existing device 1201. The apparatus 1200 adds one or more features as described with reference to FIGS. 1-11 in various embodiments of the invention.

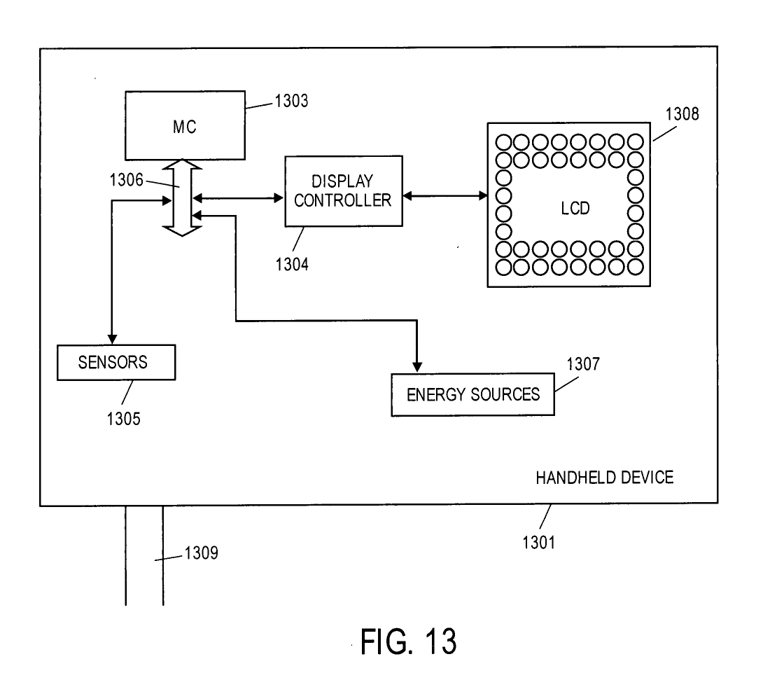

FIG. 13 shows an electric architecture for a handheld device such as device 900. The device 1301 shown in FIG. 13 includes an LCD display 1308 having multiple rows and columns of pixels. The output of display may be the same as or similar to the output of display 800. The display 1308 is coupled to a programmable or programmed micro controller 1303 through a display controller 1304; it will be appreciated that the display controller 1304 may be eliminated if the micro controller performs the display updating functions of the display controller. The micro controller 1303 is coupled to sensors 1305 and to energy sources 1307 through a bus 1306. The sensors 1305 may be electrical skin contact sensors (such as, e.g., electrodes 503d and 503e), or pressure sensors which detect a pressure above or below atmospheric pressure, or skin temperature sensors, or skin color sensors or a combination of these (and other) sensors. The energy sources 1307 may be multiple light sources or radio frequency electrical electrodes or other types of energy sources described herein or a combination of these sources. The device 1301 also includes a cable 1309, which is similar to cable 905 (attached to handle 906) of the device 900 of FIG. 9. The cable provides power to the handheld from a separate power supply (which may be bulky and thus not practical to hold in a hand), and the cable also provides vacuum and air pressures from a separate (potentially bulky) vacuum pump and air pump. The device 900 also includes manual controls such as a pneumatic adjustment control 903 (allowing the vacuum to be adjusted) and a power adjustment control 904 (allowing the power of a treatment to be adjusted manually by an operator). The device 900 also includes a disposable tip 902 which may be a detachable membrane such as membrane 301 which attaches to the treatment face of the body of the device 900.

The micro controller 1303 may be programmed to operate the device in one or more of the methods described herein. For example, the micro controller 1303 may receive signals from a skin color sensor 1305 which causes the micro controller 1303 to automatically adjust (without any user input or intervention) the power level of the energy sources; the handheld display can then be updated to show that the power level has been changed (and this may be noticed by the operator who can override the changed power setting). The skin color sensor(s) may also be used to detect the return of blood pushed away by an object protruding within the recess of the device; upon detecting this change in skin color from signals from the skin color sensor, the micro controller shuts off the power to the energy sources in one embodiment of the invention, and another cycle (e.g., as shown in FIG. 2a) may be performed to continue the treatment at the same treatment site. The micro controller 1303 may also receive signals from a skin temperature sensor 1305 which causes the micro controller 1303 to automatically adjust (without any user input or intervention) the power level of the energy sources; if, for example, the skin temperature becomes too hot, the micro controller may completely turn off the power to the energy sources in order to protect the patient's skin.

The micro controller 1303 may also receive signals from a pressure sensor which indicates that the device has been presses against the skin at a desired treatment site, thereby creating a seal between the device and the skin; the resulting pressure change (due to this seal) in the recess is detected, and the micro controller begins, automatically, a desired treatment (at either predetermined settings previously entered by an operator or automatically based on skin color sensor signals and settings previously entered by an operator). In this case, the micro controller may cause an object (e.g., object 401) to press against the skin and cause the vacuum to be generated and then apply energy from the energy sources before the blood returns to the treatment. Pressing the object against the skin and generating a vacuum may be concurrent (completely overlapped in time) or partially overlapping in time or sequential with no overlap in time. The micro controller 1303 may use a timer to determine when the blood returns (to a normal concentration level after having been pushed away) or may use signals from a skin color sensor; the timer may be started upon pushing with the protruding object, and the elapsed time may be counted. In this way, the micro controller can assure that the energy is applied in the time period (e.g., 100 m sec) before the blood returns to a normal concentration. If the object which pushes the blood away is moveable, the micro controller may control its movement.

The subject invention has been described with reference to numerous details set forth herein and the accompanying drawings. This description and accompanying drawings are illustrative of the invention and are not to be construed as limiting the invention. It will be evident that various modifications may be made thereto without departing from the broader spirit and scope of the invention as set forth in the following claims.

1. A method that treats a target, comprising:
applying a device to an area of biological external tissue having a target;
applying a positive pressure on said area;
applying a negative pressure on said area; and
applying an energy to said area before the blood concentration in said area returns to a normal state.

2. The method in claim 1, further comprising: keeping track of the number of uses of said device.

3. The method in claim 1, in which said positive pressure is a gas.

4. The method in claim 3, in which said gas is a cooling gas that is applied before applying energy.

5. The method in claim 1, in which said energy originates from a source that is not exposed to said positive pressure and said negative pressure.

6. The method in claim 1, wherein said energy is at least one of incoherent light, coherent light, radio frequency, or ultrasound.

7. The method in claim 1, wherein said energy is a radio frequency and a coherent light.

8. The method in claim 1, further comprising: generating a peripheral vacuum seal to keep said device on said area.

9. The method in claim 1, further comprising: applying an electrical current to said area before the blood concentration in said area returns to at least a normal state or higher concentration than normal.

10. The method in claim 1, further comprising: measuring color of said biological external tissue.

11. The method in claim 1, further comprising: measuring temperature of said biological external tissue.

12. The method in claim 1, further comprising: displaying at least one measurement of a sensor on said device.

13. The method in claim 1, further comprising:
providing power to said device; and
generating said positive pressure and said negative pressure through a pressure source connected to said device through a cable element.

14. The method in claim 1, further comprising: regulating the strength of said energy.

15. The method in claim 1, further comprising: preventing said biological external tissue that is outside said device from stretching.

16. The method in claim 1, further comprising: pushing away blood inside said biological external tissue.

17. A method that treats a target, comprising:
applying a device to an area of biological external tissue having a target;
applying a negative pressure on said area and bringing said biological external tissue into contact with a protruding object of said device that is above said area; and
applying an energy to said area before the blood concentration in said area returns to at least a normal state.

18. The method in claim 17, further comprising: keeping track of the number of uses of said device.

19. The method in claim 17, in which said negative pressure is a vacuum, and wherein said protruding object is substantially transparent to said energy.

20. The method in claim 17, in which said energy originates from a source that is not exposed to said pressure.

21. The method in claim 17, wherein said energy is at least one of incoherent light, coherent light, radio frequency, or ultrasound.

22. The method in claim 17, wherein said energy is a radio frequency and a coherent light.

23. The method in claim 17, further comprising: generating a peripheral vacuum seal to keep said device on said area.

24. The method in claim 17, further comprising: applying an electrical current to said area before the blood concentration in said area returns to at least a normal state.

25. The method in claim 17, further comprising: measuring color of said biological external tissue.

26. The method in claim 17, further comprising: measuring temperature of said biological external tissue.

27. The method in claim 17, further comprising: displaying at least one measurement of a sensor on said device.

28. The method in claim 17, further comprising:
providing power to said device; and
generating said negative pressure through a pressure source connected to said device through a cable element.

29. The method in claim 17, further comprising: regulating the strength of said energy.

30. The method in claim 17, further comprising: preventing said biological external tissue that is outside said device from stretching.

31. The method in claim 17, further comprising: pushing away blood inside said biological external tissue.

32. A method that treats a target, comprising:
applying a device to an area of biological external tissue having a target;
applying a first positive pressure on said area;
applying a negative pressure on said area and bringing said biological external tissue into contact with said device that is above said area;
applying an energy to said area before the blood concentration in said area returns to at least a normal state; and
applying a second positive pressure on said area to allow said device to be released from said area.

33. The method in claim 32, further comprising: keeping track of the number of uses of said device.

34. The method in claim 32, in which said first positive pressure and said second positive pressure is a gas.

35. The method in claim 34, in which said gas is a cooling gas that is applied before applying energy.

36. The method in claim 32, in which said energy originates from a source that is not exposed to said first positive pressure, said negative pressure, and said second positive pressure.

37. The method in claim 32, wherein said energy is at least one of incoherent light, coherent light, radio frequency, or ultrasound.

38. The method in claim 32, wherein said energy is a radio frequency and a coherent light.

39. The method in claim 32, further comprising: generating a peripheral vacuum seal to keep said device on said area.

40. The method in claim 32, in which said area of biological external tissue is inside a peripheral vacuum of the device and skin.

41. The method in claim 32, further comprising: applying an electrical current to said area before the blood concentration in said area returns to at least a normal state.

42. The method in claim 32, further comprising: measuring color of said biological external tissue.

43. The method in claim 32, further comprising: measuring temperature of said biological external tissue.

44. The method in claim 32, further comprising: displaying at least one measurement of a sensor on said device.

45. The method in claim 32, further comprising:
providing power to said device; and
generating said first positive pressure, said negative pressure, and said second positive pressure through a pressure source connected to said device through a cable element.

46. The method in claim 32, further comprising: regulating the strength of said energy.

47. The method in claim 32, further comprising: preventing said biological external tissue that is outside said device from stretching.

48. The method in claim 32, further comprising: pushing away blood inside said biological external tissue.

49. A device that applies energy to biological external tissue, said device comprising:
a body having a surface which is applied to said biological external tissue;
at least two different energy sources coupled to said body, said at least two different energy sources being used to delivery energy to said biological external tissue; and
a pressure conduit coupled to said surface, said pressure conduit generates a negative pressure in an area that includes said biological external tissue.

50. The device in claim 49, further comprising a processor on said body that keeps track of the number of uses of said device.

51. The device in claim 49, in which said pressure conduit also generates a positive pressure in an area that includes said biological external tissue.

52. The device in claim 50, in which said positive pressure is a gas.

53. The device in claim 51, in which said gas is a cooling gas that is applied before applying energy.

54. The device in claim 49, in which said different energy sources are not exposed to said pressure.

55. The device in claim 49, wherein said pressure conduit generates a peripheral vacuum seal.

56. The device in claim 49, wherein said energy is at least one incoherent light, coherent light, radio frequency, uniform light, or ultrasound.

57. The device in claim 49, wherein said energy is a radio frequency and a coherent light.

58. The device in claim 49, further comprising a pair of electrodes connected to opposite sides of said body that applies an electrical current through said biological external tissue.

59. The device in claim 49, further comprising a skin color sensor connected to said body that measures color of said biological external tissue.

60. The device in claim 49, further comprising a skin temperature sensor connected to said body that measures temperature of said biological external tissue.

61. The device in claim 49, further comprising a display element on said body that displays at least one measurement of a sensor on said body.

62. The device in claim 49, further comprising a pressure source, said pressure source providing power to said body and generating said pressure, said pressure source connected to said body through a cable element.

63. The device in claim 49, further comprising a controller on said body that regulates the strength of said energy.

64. The device in claim 49, further comprising: a tapered outer wall on the periphery of said device that prevents said biological external tissue that is outside said device from stretching.

65. The device in claim 49, further comprising: an object coupled to said body that pushes away blood inside said biological external tissue.

66. A device that applies energy to biological external tissue, said device comprising:
a body having a surface which is applied to said biological external tissue;
a pair of electrodes connected to opposite sides of said body that applies an electrical current through said biological external tissue; and
a pressure conduit coupled to said body, said pressure conduit to generate a pressure in an area that includes said biological external tissue, and a protruding object of said device that is above said biological external tissue and is to be brought into contact with said biological external tissue.

67. The device in claim 66, further comprising a processor on said body that keeps track of the number of uses of said device.

68. The device in claim 66, in which said pressure conduit generates a positive pressure in an area that includes said biological external tissue.

69. The device in claim 66, in which said pressure conduit generates a negative pressure in an area that includes said biological external tissue.

70. The device in claim 66, in which said pressure conduit generates a positive pressure and a negative pressure in an area that includes said biological external tissue.

71. The device in claim 67, in which said positive pressure is a gas.

72. The device in claim 70, in which said gas is a cooling gas that is applied before applying said electrical current.

73. The device in claim 66, in which said electrodes originate from a source that is not exposed to said pressure.

74. The device in claim 66, further comprising a skin color sensor connected to said body that measures color of said biological external tissue.

75. The device in claim 66, further comprising a skin temperature sensor connected to said body that measures temperature of said biological external tissue.

76. The device in claim 66, further comprising a display element on said body that displays at least one measurement of a sensor on said body.

77. The device in claim 66, further comprising a pressure source, said pressure source providing power to said body and generating said pressure, said pressure source connected to said body through a cable element.

78. The device in claim 66, further comprising a controller on said body that regulates the strength of said electrical current.

79. The device in claim 66, further comprising: a tapered outer wall on the periphery of said device that prevents said biological external tissue that is outside said device from stretching.

80. The device in claim 66, wherein said pressure conduit generates a peripheral vacuum seal.

81. A device that applies energy to biological external tissue, said device comprising:
a body having a surface which is applied to said biological external tissue;
an energy source coupled to said body to delivery energy to said biological external tissue; and
a pressure conduit coupled to said surface, said pressure conduit that generates a positive pressure comprising a gas in an area that includes said biological external tissue, said gas pushing blood away in said area.

82. The device in claim 81, further comprising a processor on said body that keeps track of the number of uses of said device.

83. The device in claim 81, in which said pressure conduit also generates at a different time a negative pressure in an area that includes said biological external tissue.

84. The device in claim 81, in which said gas is a cooling gas that is applied before applying energy.

85. The device in claim 81, wherein said energy is at least one of incoherent light, coherent light, radio frequency, uniform light, or ultrasound.

86. The device in claim 81, wherein said energy is a radio frequency and a coherent light.

87. The device in claim 81, wherein said pressure conduit generates a peripheral vacuum seal.

88. The device in claim 81, further comprising a pair of electrodes connected to opposite sides of said body that applies an electrical current through said biological external tissue.

89. The device in claim 81, further comprising a skin color sensor connected to said body that measures color of said biological external tissue.

90. The device in claim 81, further comprising a skin temperature sensor connected to said body that measures temperature of said biological external tissue.

91. The device in claim 81, further comprising a display element on said body that displays at least one measurement of a sensor on said body.

92. The device in claim 81, further comprising a pressure source, said pressure source providing power to said body and generating said pressure, said pressure source connected to said body through a cable element.

93. The device in claim 81, further comprising a controller on said body that regulates the strength of said energy.

94. The device in claim 81, further comprising: a tapered outer wall on the periphery of said device that prevents said biological external tissue that is outside said device from stretching.

95. The device in claim 81, further comprising: an object coupled to said body that pushes away blood inside said biological external tissue.

96. A device that applies energy to biological external tissue, said device comprising:
a body having a surface which is applied to said biological external tissue;
an energy source coupled to said body to delivery energy to said biological external tissue; and
a skin temperature sensor connected to said body that measures temperature of said biological external tissue, wherein said skin temperature sensor is a capacitance sensor.

97. The device in claim 96, further comprising a pressure conduit coupled to said surface, said pressure conduit that generates a pressure in an area that includes said biological external tissue.

98. The device in claim 96, further comprising a processor on said body that keeps track of the number of uses of said device.

99. The device in claim 97, in which said pressure conduit generates a positive pressure in an area that includes said biological external tissue.

100. The device in claim 97, in which said pressure conduit generates a negative pressure in an area that includes said biological external tissue.

101. The device in claim 97, in which said pressure conduit generates a positive pressure and a negative pressure in an area that includes said biological external tissue.

102. The device in claim 97, wherein said pressure conduit generates a peripheral vacuum seal.

103. The device in claim 99, in which said positive pressure is a gas.

104. The device in claim 103, in which said gas is a cooling gas that is applied before applying energy.

105. The device in claim 97, in which said energy source is not exposed to said pressure.

106. The device in claim 97, wherein said energy is at least one of incoherent light, coherent light, radio frequency, uniform light, or ultrasound.

107. The device in claim 97, wherein said energy is a radio frequency and a coherent light.

108. The device in claim 96, further comprising a pair of electrodes connected to opposite sides of said body that applies an electrical current through said biological external tissue.

109. The device in claim 96, further comprising a skin color sensor connected to said body that measures color of said biological external tissue.

110. The device in claim 96, further comprising a display element on said body that displays at least one measurement of a sensor on said body.

111. The device in claim 97, further comprising a pressure source, said pressure source providing power to said body and generating said pressure, said pressure source connected to said body through a cable element.

112. The device in claim 96, further comprising a controller on said body that regulates the strength of said energy.

113. The device in claim 96, further comprising: a tapered outer wall on the periphery of said device that prevents said biological external tissue that is outside said device from stretching.

114. The device in claim 96, further comprising: an object coupled to said body that pushes away blood inside said biological external tissue.

115. A device that applies energy to biological external tissue, said device comprising:
a body having a surface which is applied to said biological external tissue;
an energy source coupled to said body to delivery energy to said biological external tissue; and
a skin color sensor coupled to said body that measures color of said biological external tissue.

116. The device in claim 115, further comprising a pressure conduit coupled to said surface, said pressure conduit that generates a pressure in an area that includes said biological external tissue.

117. The device in claim 115, further comprising a processor on said body that keeps track of the number of uses of said device.

118. The device in claim 115, wherein skin color sensor is a skin capacitance sensor.

119. The device in claim 116, in which said pressure conduit generates a positive pressure in an area that includes said biological external tissue.

120. The device in claim 116, in which said pressure conduit generates a negative pressure in an area that includes said biological external tissue.

121. The device in claim 116, in which said pressure conduit generates a positive pressure and a negative pressure in an area that includes said biological external tissue.

122. The device in claim 116, wherein said pressure conduit generates a peripheral vacuum seal.

123. The device in claim 119, in which said positive pressure is a gas.

124. The device in claim 123, in which said gas is a cooling gas that is applied before applying energy.

125. The device in claim 116, in which said energy source is not exposed to said pressure.

126. The device in claim 115, wherein said energy is at least one of incoherent light, coherent light, radio frequency, uniform light, or ultrasound.

127. The device in claim 115, wherein said energy is a radio frequency and a coherent light.

128. The device in claim 115, further comprising a pair of electrodes connected to opposite sides of said body that applies an electrical current through said biological external tissue.

129. The device in claim 115, further comprising a skin temperature sensor connected to said body that measures temperature of said biological external tissue.

130. The device in claim 115, further comprising a display element on said body that displays at least one measurement of a sensor on said body.

131. The device in claim 116, further comprising a pressure source, said pressure source providing power to said body and generating said pressure, said pressure source connected to said body through a cable element.

132. The device in claim 115, further comprising a controller on said body that regulates the strength of said energy.

133. The device in claim 115, fuirther comprising: a tapered outer wall on the periphery of said device that prevents said biological external tissue that is outside said device from stretching.

134. The device in claim 115, further comprising: an object coupled to said body that pushes away blood inside said biological external tissue.

135. A device that applies energy to biological external tissue, said device comprising:
a body having a surface which is applied to said biological external tissue;
an object that pushes away blood inside said biological external tissue, said object being coupled to said body and being disposed within a recess of said body;
an energy source coupled to said body to delivery energy to said biological external tissue; and
a pressure conduit coupled to said surface, said pressure conduit that generates a pressure in an area that includes said biological external tissue.

136. The device in claim 135, in which said pressure conduit generates a vacuum to the periphery of said object and wherein said object is solid.

137. The device in claim 135, further comprising a processor on said body that keeps track of the number of uses of said device.

138. The device in claim 135, in which said pressure conduit generates a positive pressure in an area that includes said biological external tissue.

139. The device in claim 135, in which said pressure conduit generates a negative pressure in an area that includes said biological external tissue and within said recess.

140. The device in claim 135, in which said pressure conduit generates a positive pressure and a negative pressure in an area that includes said biological external tissue.

141. The device in claim 138, in which said positive pressure is a gas.

142. The device in claim 141, in which said gas is a cooling gas that is applied before applying energy.

143. The device in claim 135, in which said energy source is not exposed to said pressure.

144. The device in claim 135, wherein said energy is at least one of incoherent light, coherent light, radio frequency, uniform light, or ultrasound.

145. The device in claim 135, wherein said energy is a radio frequency and a coherent light.

146. The device in claim 135, wherein said pressure conduit generates a peripheral vacuum seal.

147. The device in claim 135, further comprising a pair of electrodes connected to opposite sides of said body that applies an electrical current through said biological external tissue.

148. The device in claim 135, further comprising a skin color sensor connected to said body that measures color of said biological external tissue.

149. The device in claim 135, further comprising a skin temperature sensor connected to said body that measures temperature of said biological external tissue.

150. The device in claim 135, further comprising a display element on said body that displays at least one measurement of a sensor on said body.

151. The device in claim 135, further comprising a pressure source, said pressure source providing power to said body and generating said pressure, said pressure source connected to said body through a cable element.

152. The device in claim 135, further comprising a controller on said body that regulates the strength of said energy.

153. The device in claim 135, further comprising: a tapered outer wall on the periphery of said device that prevents said biological external tissue that is outside said device from stretching.

154. A device that applies energy to biological external tissue, said device comprising:
a handheld body having a surface which is applied to said biological external tissue;
a display element on said body that displays at least one parameter with respect to a treatment of said biological external tissue, said display element having rows and columns of pixels controlled by a display controller; and
an energy source coupled to said body to delivery energy to said biological external tissue.

155. The device in claim 154, further comprising a pressure conduit coupled to said surface, said pressure conduit that generates a pressure in an area that includes said biological external tissue.

156. The device in claim 154, further comprising a processor on said body that keeps track of the number of uses of said device.

157. The device in claim 155, in which said pressure conduit generates a positive pressure in an area that includes said biological external tissue.

158. The device in claim 155, in which said pressure conduit generates a negative pressure in an area that includes said biological external tissue.

159. The device in claim 155, in which said pressure conduit generates a positive pressure and a negative pressure in an area that includes said biological external tissue.

160. The device in claim 155, wherein said pressure conduit generates a peripheral vacuum seal.

161. The device in claim 157, in which said positive pressure is a gas.

162. The device in claim 160, in which said gas is a cooling gas that is applied before applying energy.

163. The device in claim 155, in which said energy source is not exposed to said pressure.

164. The device in claim 154, wherein said energy is at least one of incoherent light, coherent light, radio frequency, uniform light, or ultrasound.

165. The device in claim 154, wherein said energy is a radio frequency and a coherent light.

166. The device in claim 154, further comprising a pair of electrodes connected to opposite sides of said body that applies an electrical current through said biological external tissue.

167. The device in claim 154, further comprising a skin color sensor connected to said body that measures color of said biological external tissue.

168. The device in claim 154, further comprising a skin temperature sensor connected to said body that measures temperature of said biological external tissue.

169. The device in claim 155, further comprising a pressure source, said pressure source providing power to said body and generating said pressure, said pressure source connected to said body through a cable element.

170. The device in claim 154, further comprising a controller on said body that regulates the strength of said energy.

171. The device in claim 154, further comprising: a tapered outer wall on the periphery of said device that prevents said biological external tissue that is outside said device from stretching.

172. The device in claim 154, further comprising: an object coupled to said body that pushes away blood inside said biological external tissue.

173. A device that applies energy to biological external tissue, said device comprising:
a body having a surface which is applied to said biological external tissue;
a pressure conduit coupled to said surface, said pressure conduit that generates a negative pressure in an area that includes said biological external tissue; and
an energy source coupled to said body to delivery energy to said biological external tissue, said energy source not exposed to said negative pressure.

174. The device in claim 173, further comprising a processor on said body that keeps track of the number of uses of said device.

175. The device in claim 173, in which said pressure conduit also generates a positive pressure in an area that includes said biological external tissue.

176. The device in claim 173, in which said negative pressure is a vacuum, and wherein light from said energy source is conveyed without a wave guide or optical fiber.

177. The device in claim 175, in which said positive pressure is a gas.

178. The device in claim 177, in which said gas is a cooling gas that is applied before applying energy.

179. The device in claim 173, in which said energy source is shielded from said negative pressure by a transparent cover which is adjacent to said surface.

180. The device in claim 173, wherein said energy is at least one of incoherent light, coherent light, radio frequency, uniform light, or ultrasound.

181. The device in claim 173, wherein said energy is a radio frequency and a coherent light.

182. The device in claim 173, wherein said pressure conduit generates a peripheral vacuum seal.

183. The device in claim 173, further comprising a pair of electrodes connected to opposite sides of said body that applies an electrical current through said biological external tissue.

184. The device in claim 173, further comprising a skin color sensor connected to said body that measures color of said biological external tissue.

185. The device in claim 173, further comprising a skin temperature sensor connected to said body that measures temperature of said biological external tissue.

186. The device in claim 173, further comprising a display element on said body that displays at least one measurement of a sensor on said body.

187. The device in claim 173, further comprising a pressure source, said pressure source providing power to said body and generating said pressure, said pressure source connected to said body through a cable element.

188. The device in claim 173, further comprising a controller on said body that regulates the strength of said energy.

189. The device in claim 173, further comprising: a tapered outer wall on the periphery of said device that prevents said biological external tissue that is outside said device from stretching.

190. The device in claim 173, further comprising: an object coupled to said body that pushes away blood inside said biological external tissue.

191. A device that applies energy to biological external tissue, said device comprising:
a body having a surface which is applied to said biological external tissue;
a first conduit that applies a vacuum to a border region of biological external tissue which surrounds a portion of biological external tissue;
a second conduit that applies a vacuum to said portion of biological external tissue; and
an energy source coupled to said body to deliver energy to said portion of biological external tissue.

192. The device in claim 191, further comprising a processor on said body that keeps track of the number of uses of said device.

193. The device in claim 191, in which said first conduit and said second conduit also generate a positive pressure in an area that includes said biological external tissue.

194. The device in claim 191, in which said first conduit and said second conduit generate a positive pressure and a negative pressure in an area that includes said biological external tissue.

195. The device in claim 193, in which said positive pressure is a gas.

196. The device in claim 195, in which said gas is a cooling gas that is applied before applying energy.

197. The device in claim 191, in which said energy source is not exposed to said pressure.

198. The device in claim 191, wherein said energy is at least one of incoherent light, coherent light, radio frequency, uniform light, or ultrasound, and wherein said device further comprises a protruding object that is brought into contact with said portion of biological external tissue.

199. The device in claim 191, wherein said energy is a radio frequency and a coherent light.

200. The device in claim 191, further comprising a pair of electrodes connected to opposite sides of said body that applies an electrical current through said biological external tissue.

201. The device in claim 191, further comprising a skin color sensor connected to said body that measures color of said biological external tissue.

202. The device in claim 191, further comprising a skin temperature sensor connected to said body that measures temperature of said biological external tissue.

203. The device in claim 191, further comprising a display element on said body that displays at least one measurement of a sensor on said body.

204. The device in claim 191, further comprising a pressure source, said pressure source providing power to said body and generating said pressure, said pressure source connected to said body through a cable element.

205. The device in claim 191, further comprising a controller on said body that regulates the strength of said energy.

206. The device in claim 191, further comprising: a tapered outer wall on the periphery of said device that prevents said biological external tissue that is outside said device from stretching.

207. An apparatus that attaches to an existing device that applies energy to biological external tissue, said apparatus comprising:
a handheld body having a surface which is applied to said biological external tissue;
a display element on said body that displays at least one parameter with respect to a treatment of said biological external tissue, said display element having rows and columns of pixels controlled by a display controller; and
an energy source coupled to said body to delivery energy to said biological external tissue.

208. The apparatus in claim 207, further comprising a pressure conduit coupled to said surface, said pressure conduit that generates a pressure in an area that includes said biological external tissue.

209. The apparatus in claim 207, further comprising a processor on said body that keeps track of the number of uses of said apparatus.

210. The apparatus in claim 208, in which said pressure conduit generates a positive pressure in an area that includes said biological external tissue.

211. The apparatus in claim 208, in which said pressure conduit generates a negative pressure in an area that includes said biological external tissue.

212. The apparatus in claim 208, in which said pressure conduit generates a positive pressure and a negative pressure in an area that includes said biological external tissue.

213. The apparatus in claim 208, wherein said pressure conduit generates a peripheral vacuum seal.

214. The apparatus in claim 210, in which said positive pressure is a gas.

215. The apparatus in claim 214, in which said gas is a cooling gas that is applied before applying energy.

216. The apparatus in claim 208, in which said energy source is not exposed to said pressure.

217. The apparatus in claim 207, wherein said energy is at least one of incoherent light, coherent light, radio frequency, uniform light, or ultrasound.

218. The apparatus in claim 207, wherein said energy is a radio frequency and a coherent light.

219. The apparatus in claim 207, further comprising a pair of electrodes connected to opposite sides of said body that applies an electrical current through said biological external tissue.

220. The apparatus in claim 207, further comprising a skin color sensor connected to said body that measures color of said biological external tissue.

221. The apparatus in claim 207, further comprising a skin temperature sensor connected to said body that measures temperature of said biological external tissue.

222. The apparatus in claim 208, further comprising a pressure source, said pressure source providing power to said body and generating said pressure, said pressure source connected to said body through a cable element.

223. The apparatus in claim 207, further comprising a controller on said body that regulates the strength of said energy.

224. The apparatus in claim 207, further comprising: a tapered outer wall on the periphery of said apparatus that prevents said biological external tissue that is outside said apparatus from stretching.

225. The apparatus in claim 207, further comprising: an object coupled to said body that pushes away blood inside said biological external tissue.

226. A method for treating a target with a device, said method comprising:
applying said device to an area of biological external tissue having said target;
applying a negative pressure on said area;
applying an energy to said area; and
applying a positive pressure on said area and then removing said device from said area.

227. A method as in claim 226 wherein said energy is applied after said negative pressure is applied and wherein said positive pressure is applied after said energy is applied.
